(19) United States
(12) Patent Application Publication
(10) Pub. No.: US 2024/0057963 A1
(43) Pub. Date: Feb. 22, 2024

(54) Wearable Auscultation Device

(71) Applicant: Senti Tech Limited, Liverpool (GB)

(72) Inventors: Paul Rimmer, Waterloo, Liverpool (GB); Philip Alton, Liverpool (GB); James Morley-Alton, Longridge, Preston (GB); Simon Levi, Wirral (GB)

(21) Appl. No.: 18/271,743

(22) PCT Filed: Jan. 24, 2022
(86) PCT No.: PCT/GB2022/050184

§ 371 Date: Jul. 11, 2023

(30) Foreign Application Priority Data
Jan. 25, 2021 (GB) 2100957.6

(51) Int. Cl. A61B 7/00; A61B 5/024

(52) U.S. Cl. CPC A61B 7/003; A61B 5/024; A61B 2560/0209; A61B 2560/0247; A61B 2560/0462; A61B 2560/045; A61B 2562/0204; A61B 2562/046

(57) ABSTRACT

The invention provides a wearable auscultation device (10) comprising a wearable sensor support (12) configured to be worn on a user's torso and to carry a plurality of auscultation modules (14), positioning them in use at respective auscultation sites on the torso and retaining them in contact with the user's skin. At least one of the auscultation modules (14) comprises: a body (86) of elastomer material providing a contact face (39) for contact with the user's skin; an auscultation microphone (82) disposed within the body (86) of elastomer material and directed toward the contact face to sense sound waves conducted through the elastomer material.

The invention concerns a wearable auscultation device.

Auscultation has for centuries been an important technique in diagnosis and assessment of a range of medical conditions, especially conditions relating to the circulatory and respiratory systems. At one time clinicians would place an ear against the patient's chest to hear sounds from it, especially those created by the heart and lungs. The stethoscope supplanted this practice, enabling the clinician to listen easily to sounds at a number of auscultation sites, typically on the torso. Traditional stethoscopes use a resonator placed against the patient and a pair of flexible tubes to conduct sound to earpieces. Electronic stethoscopes having a microphone placed upon the patient to detect sounds are now available.

A clinician using a stethoscope typically only listens for a brief period. Given sufficient skill on the part of the clinician, this brief snapshot is sufficient to identify a number of clinical conditions. But there are significant potential benefits in performing auscultation over a more prolonged period of time. In this way conditions that manifest intermittently or only under certain conditions may be detected, changes over time of existing conditions may be monitored, predictions may be made—and warnings given to the patient—if a certain condition worsens, and so on.

There are known wearable devices for carrying out a form of electronic auscultation.

US2019/231262A1, Nasry, discloses a garment having a range of clinical sensors and other apparatus including a plurality of auscultation acoustic sensor devices which may take the form of digital microphones or electronic stethoscopes. Little detail is provided about the sensor devices themselves. WO2019/161277A1, Northwestern University et al, contains ideas for the construction and use of a range of wireless medical sensors, which in some examples comprise mechano-acoustic sensors. US2018/0177483A1, Ye et al, discloses a “high frequency chest wall oscillation vest” for providing a form of percussive therapy intended to clear airways, in combination with a respiratory acoustic analysis system for sensing and analysing respiratory sounds. WO2019/241674, Strados Labs LLC, discloses a method in which motion data and audio data are both collected using sensors of a wearable device, and are analysed in conjunction. The data can be used to recognise when the user has coughed. WO2011/117862, Melman et al, discloses a garment on whose rear can be carried sensors of a range of different types, which may be piezo-electric sensors encased in silicone. GB2574040A discloses a device comprising acoustic transducers mounted in an enclosure having an acoustic port. One of the acoustic transducers serves to identify background signals and a processor is provided for removing these signals and isolating a target acoustic signal. US2019/0298269A1, Atashbar et al, discloses a stethographic device having a plurality of stethoscopes. In one example these are embedded in a memory foam pad on the back rest of a chair. Individual stethoscopes have in one example a casing with a through-hole containing a microphone which is covered by a Littmann diaphragm.

Problems remain with known electronic auscultation devices. These include:
vulnerability of auscultation devices to damage, by physical impacts and/or by contamination;
problems associated with noise in the audio signal, including unwanted noise created by movement of the auscultation device across the skin of a user;
practical problems associated with wearing a device for a protracted period, including the need to wash the device for the sake of personal hygiene, and the discomfort that may result from wearing a cumbersome device.

Providing a solution to one or more of these problems is desirable.

The present invention provides a wearable auscultation device, an auscultation module, and a method of manufacture of an auscultation device in accordance with the appended claims.

Specific embodiments of the present invention will now be described, by way of example only, with reference to the accompanying drawings, in which:

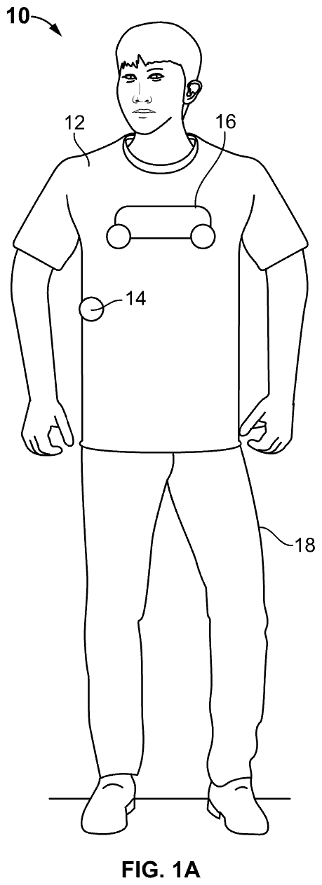

FIGS. 1a and 1b are schematic representations of a wearable auscultation device in accordance with the present invention, viewed from the front and from the rear respectively;

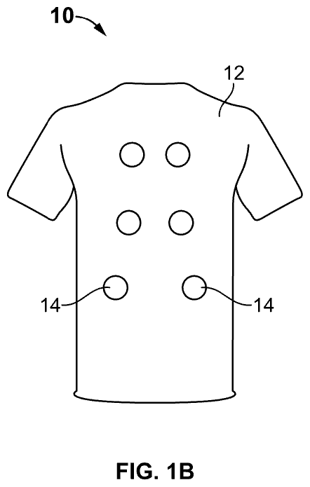

FIGS. 2a and 2b are front and rear views respectively of a further wearable auscultation device in accordance with the present invention, to be worn by a male;

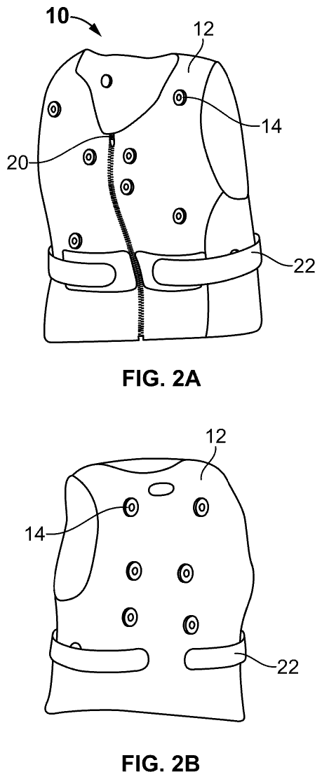

FIGS. 3a and 3b are front and rear views respectively of another wearable auscultation device in accordance with the present invention, to be worn by a female;

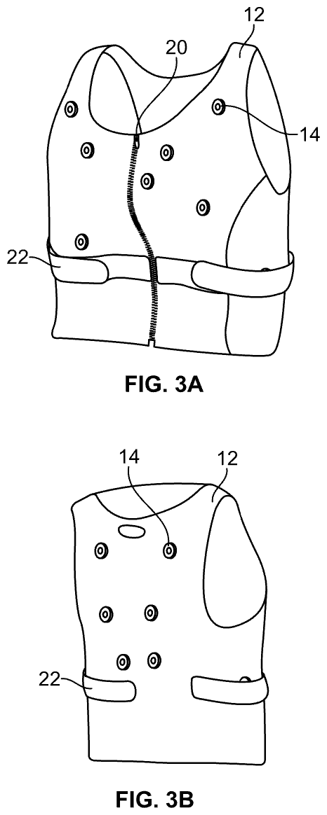

FIGS. 4a, 4b and 4c show an auscultation module and a corresponding module coupling for use in the wearable auscultation devices, these components being coupled together and viewed from the front in FIG. 4a, coupled together and viewed from one side in FIG. 4b, and de-coupled and viewed from the side in FIG. 4c;

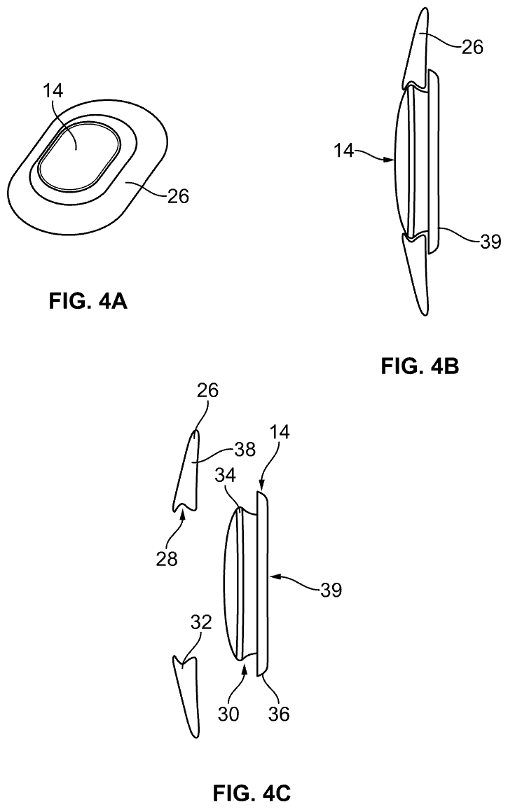

FIG. 5 is a schematic representation of functional units of a communications module 16, along with its network connections;

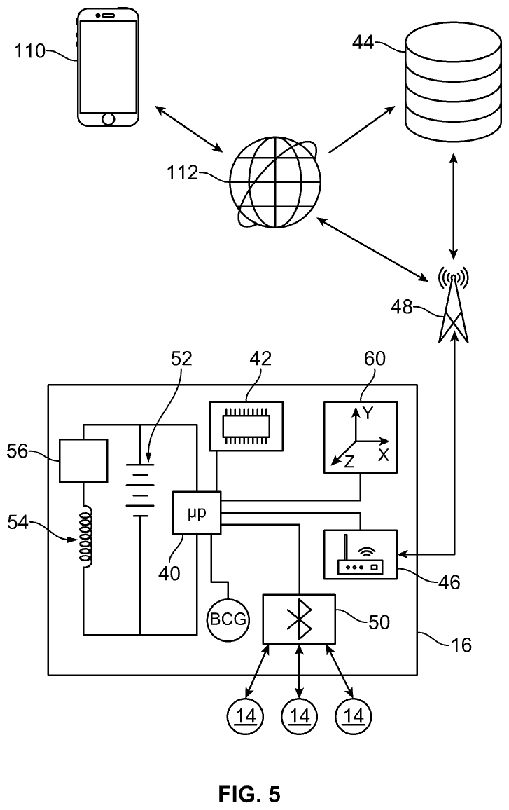

FIG. 6 is a schematic representation of major functional units of the auscultation module;

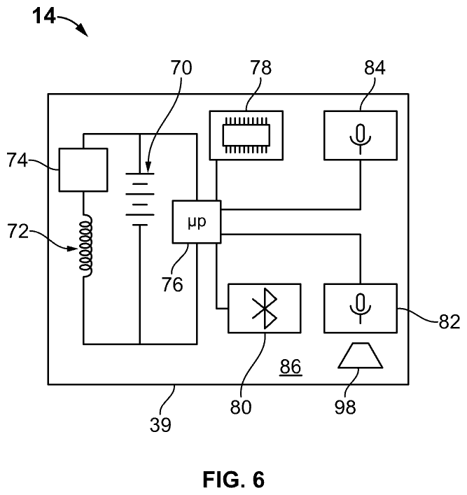

FIGS. 7a and 7b represent component parts of a mould and FIG. 7c represents the assembled mould;

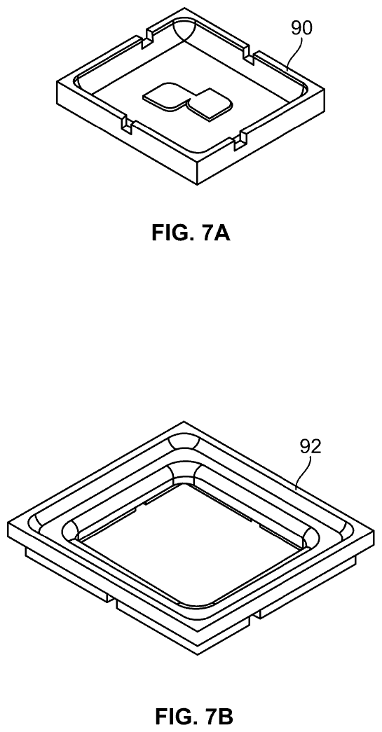

FIG. 8 is a side view of the mould, partially filled;

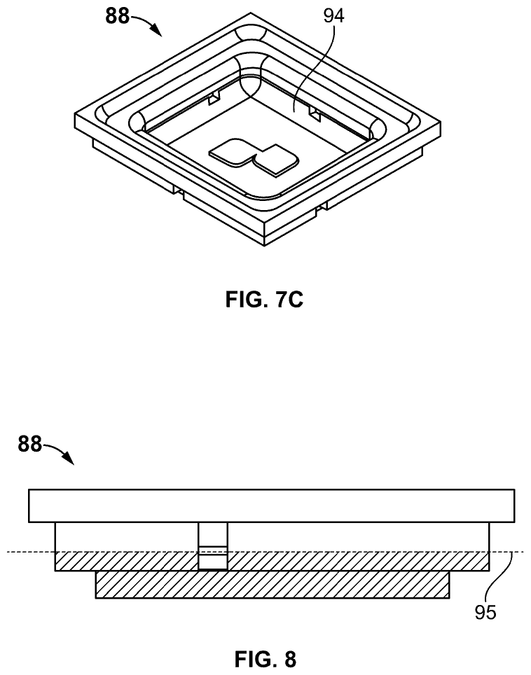

FIG. 9 is a view of the mould form above during a moulding process, a circuit board being located in the mould;

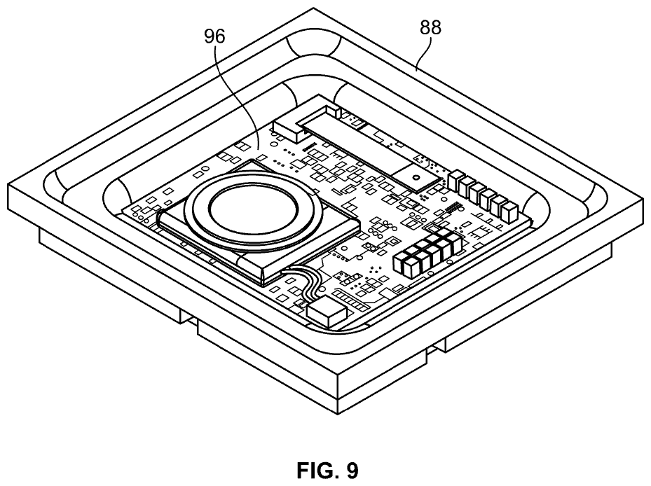

FIGS. 10a and 10b represent an auscultation module, differing in that in FIG. 10b part of the modules housing is rendered transparent, to reveal internal detail;

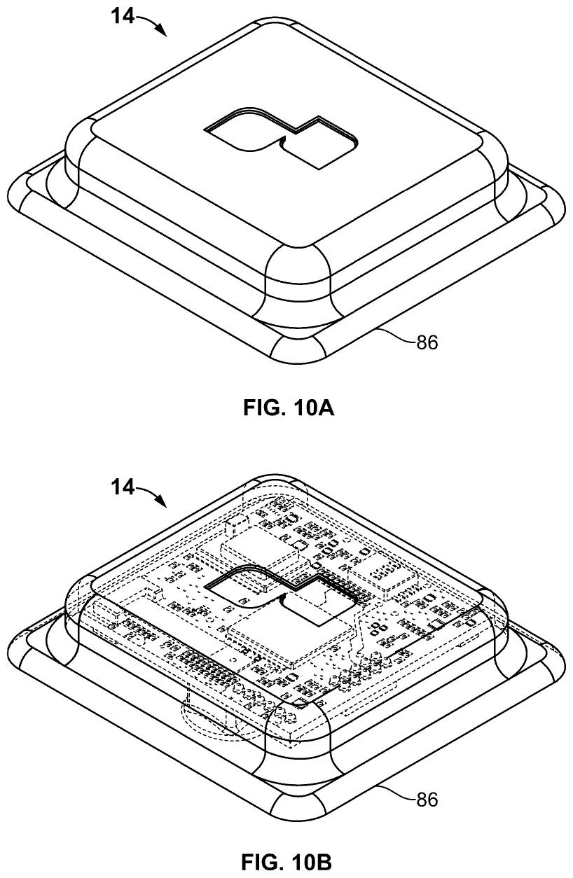

FIG. 11 depicts an acoustic element viewed from above and one side;

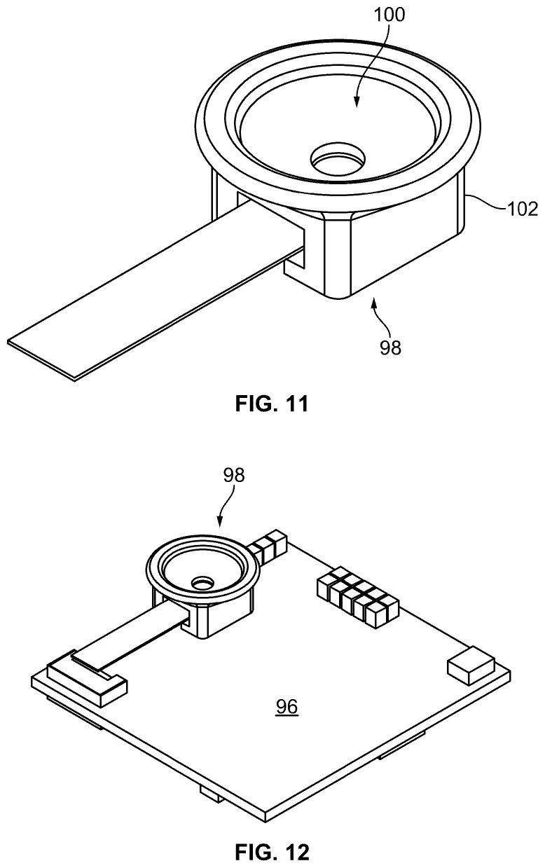

FIG. 12 depicts the acoustic element in position on a circuit board;

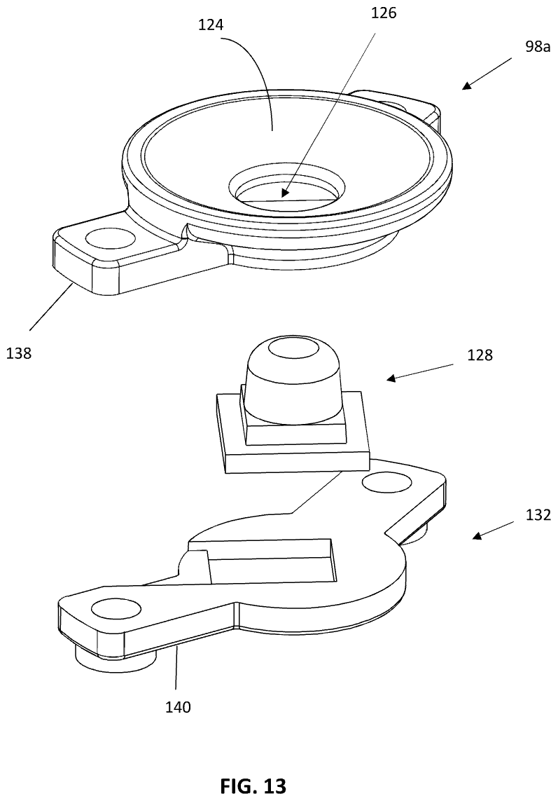

FIG. 13 is an exploded view of components of a further auscultation device embodying the present invention;

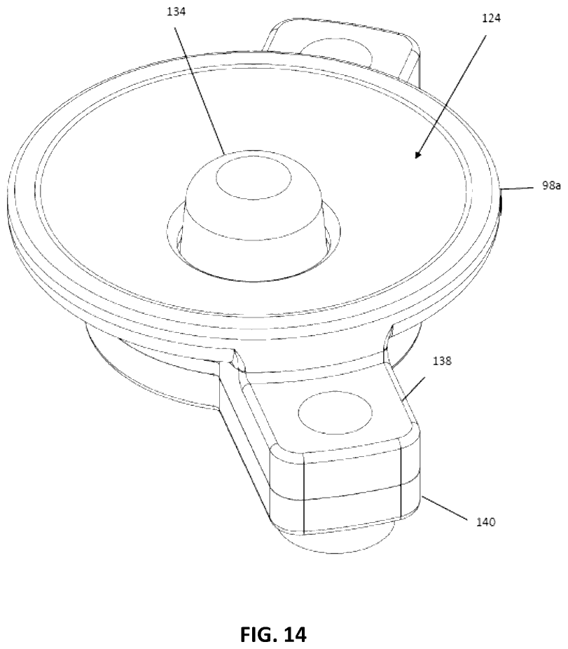

FIG. 14 shows the components of FIG. 13 assembled to one another;

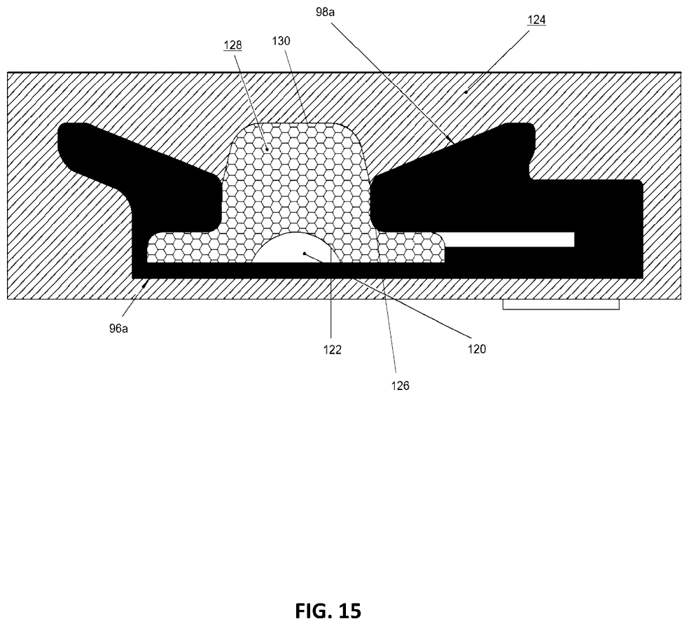

FIG. 15 is a section through parts of the further auscultation device; and

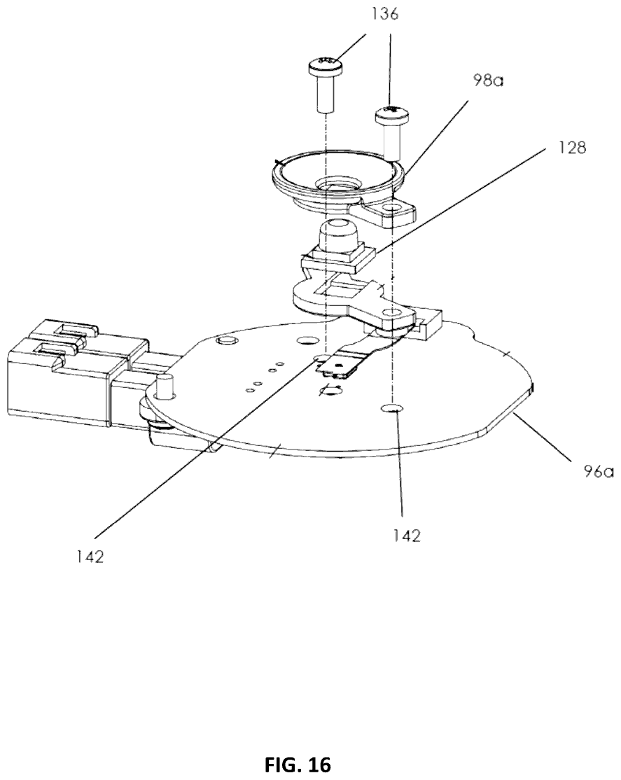

FIG. 16 is another exploded view of components of the further auscultation device.

The present invention provides, inter alia, a wearable, pan-thoracic auscultation device 10. The basic construction and operation of a first embodiment can be appreciated from FIG. 1, showing a wearable sensor carrier 12, carrying a set of auscultation modules 14 and a communications module 16.

The wearable sensor carrier 12 is to be worn upon the torso of a user 18. The function of the wearable sensor carrier 12 is to mount the auscultation modules 14 at chosen, defined locations on the user's torso, and to maintain the auscultation modules 14 in contact with the user's skin to enable effective transmission of acoustic signals. The auscultation modules 14 are positioned in the same defined locations on the user's torso each time the user 18 dons the sensor carrier 14. The wearable auscultation device 10 is intended to be worn and used for extended periods of time, so that comfort is an important design consideration, and it is desired that the wearable sensor carrier 12 should not be unduly restrictive of movement, and that it should not be unduly restrictive about the chest, which would be potentially problematic for those with respiratory conditions.

In the FIG. 1 embodiment, the wearable sensor carrier 12 comprises a shirt to be donned and doffed over the head. This example takes the form of a T-shirt comprising textile material. The textile comprises elastomer, in the present example, to enable the wearable sensor carrier to closely follow the contours of the torso and to retain the auscultation modules 14 against the skin, without exerting undue pressure. Suitable elastomer materials include spandex (also known as elastane, and sold widely under the trade mark Lycra®). For the sake of comfort and breathability, the elastomer is in the present combined with a natural yarn, more specifically cotton. The wearable sensor carrier needs to closely conform to the user's torso, and may be offered in a range of different sizes and fittings to accommodate a range of body shapes.

The number and arrangement of the auscultation modules 14 upon the wearable sensor carrier 12 may vary from one embodiment to another, but in the present embodiment the sensor locations are chosen to generally correspond to the auscultation sites at which a clinician would place a stethoscope head during a conventional clinical auscultation. Specifically, in the FIG. 1 embodiment, there are nine auscultation modules 14, three on the front of the torso and six on the rear.

FIG. 2 depicts an alternative form of the wearable sensor carrier 12 according to another embodiment of the invention, which in this example is a sleeveless jerkin having a zip fastener 20 enabling it to be donned and doffed without being drawn over the head. A belt 22 allows the user 18 to make adjustments to fit. This version is sleeveless and may be well suited to wear beneath outer clothing. The wearable sensor carrier 12 is to be offered in both male and female versions, the latter being seen in FIG. 3 and being in particular formed to accommodate the female bust while at the same time maintaining the necessary close contact of the auscultation modules 14 with the user's torso. In some embodiments, the wearable sensor carrier 12 may be configured to support the bust in a manner akin to a brassiere.

It is desirable that the wearable sensor carrier 12 should be washable by the user 18. In some embodiments the auscultation modules 14 and the communications module 18 may themselves be capable of surviving washing in water, and even in a conventional washing machine, so that they need not be removed for washing. But in other embodiments the auscultation modules 14 and the communications module 18 are removably coupled to the wearable sensor carrier 12, enabling them to be kept out of the wash. For this purpose, the wearable sensor carrier 12 may be provided with a set of auscultation module couplings 26 at the required auscultation sites, each auscultation module coupling 26 being configured to receive and mount a respective auscultation module 14.

The form of the auscultation module couplings 26 may differ from one embodiment to another, but FIG. 4 provides an example where it comprises a shallow frame defining an aperture to receive the auscultation module 14. In this example the aperture is defined by a lip 28 shaped to resiliently engage a peripheral feature 30 of the auscultation module 14, to couple to it. The lip 28 comprises a peripherally-running channel 32. The auscultation module 14 has around its periphery an upstand 34. Pushing the auscultation module 14 into place in the auscultation module coupling 26 (FIG. 4b) involves stretching the frame 26 somewhat, so that when the module is in place (FIG. 4B), the upstand 34 is received and resiliently retained in the channel 32, coupling the two parts releasably together. Removing the auscultation module 14 simply involves pulling it hard enough to overcome the resilient coupling. In this embodiment the auscultation module coupling 26 is formed from an elastomer. It may be secured to the wearable sensor carrier 12 by any suitable means—stitching, adhesive bonding, etc.

The auscultation module 14 has a brim 36 which, by engaging with an inner face 38 of the auscultation module coupling 26, prevents the auscultation module 14 from being pulled right through the frame 26. The said inner face 38 of the auscultation module coupling 26, and a contact face 39 of the auscultation module 14, provide a surface through which the arrangement rests against the user's skin. Due to its size, this surface distributes pressure across an area of the skin, avoiding excessive local pressure that could lead to discomfort.

Because the auscultation modules 14 are able to be repeatedly removed from and then re-mounted upon the wearable sensor carrier 12, the user may for example be provided with a number of wearable sensor carriers 12 (which can be economical to manufacture) and a single set of auscultation modules 14/communications module 16, allowing the sensor carrier 12 to be changed regularly, for the sake of personal hygiene and/or for replacement due to wear and tear, or even for aesthetic reasons—simply to give the user a change of what may in some cases serve as clothing.

Certain embodiments of the invention may use other means of mounting the auscultation modules 14 to the torso of the user 18, not reliant on a common wearable sensor carrier 12. For example, auscultation modules 14 could be temporarily adhered to the skin of the user, or individual auscultation modules 14 could be carried by respective belts or straps.

While it would be possible to electrically connect the auscultation modules 14 together through wiring without departing from the scope of the present invention, in the present embodiment each auscultation module 14 is self-powered and exchanges data in an un-wired manner. This data exchange preferably uses RF communication. It may exploit any suitable current or future data exchange protocol for the purpose, but in the present embodiment each auscultation module has an RF transceiver operating according to one of the Bluetooth® protocols. In the present embodiment, each auscultation module 14 exchanges data with the communications module 16. In particular, sensor data from each of the auscultation modules 14 is transmitted to the communications module 16.

The communications module 16 thus receives sensor data from each of the auscultation modules 14.

The communications module 16 comprises in the present embodiment at least one processor 40 with associated memory 42—see FIG. 5. The communications module 16 has communications apparatus enabling it to exchange data not only with the auscultation modules 14, but also with a remote server 44. Data exchange between the communications module 16 and the remote server 44 is typically carried out via one or more wide area networks. In the present embodiment, the communications module 16 is provided with a GSM modem 46 for communication via a mobile (cellular) network 48.

Additionally or alternatively, the communications module 16 may be provided with WiFi® connectivity for connection via a WiFi® local area network with a wide area network (typically the internet 112) and through that with the remote server 44. The communications module 16 also comprises a Bluetooth® interface 50 able to pair and exchange data with each of the auscultation modules 14. The memory 42 stores a lookup table indicating correspondence between the physical locations of the auscultation modules 14 in the wearable sensor carrier 12 and their respective IDs (in the present embodiment these are Bluetooth® IDs), so that each sensor signal can be associated with the relevant region of the user's thoracic anatomy. The auscultation modules 14 and the wearable sensor carrier 12 may for example be marked (e.g. numbered) to assist the user 18 in placing the auscultation modules 14 in the intended locations when re-attaching the modules.

In operation, the communications module 16 receives the sensor data from the auscultation modules 14 and passes it on to the remote server 44. It may in some embodiments carry out processing on the sensor data before passing it on. This processing may involve data compression, for example. It may serve to ensure privacy of the user 18, as will be explained below. The processing may involve encryption: in the present embodiment all data exchanged between the wearable auscultation device 10 and the remote server 44 is encrypted in suitable manner to protect the data. Public/private key encryption may for example be used.

The communications module 16 may also log sensor data in the memory 42, before or after any on-board processing thereof. A continuous connection between the communications module 16 and the remote server 44 may not be available (e.g. due to imperfect network service and coverage) and may not be desirable, on cost and bandwidth grounds. By logging sensor data on the communications module 16, loss of data due to loss of connectivity is avoided, and economical use of connectivity and bandwidth can be made (e.g. by transmitting blocks of data at intervals).

The communications module 16 comprises an on-board electrical power supply in the form of a rechargeable battery 52. In present embodiments a lithium-ion battery is used. Charging could in principle be made through electrical connections, e.g. comprising a socket for a charging lead, but in the present embodiment wireless inductive charging is used. The communications module 16 comprises an energy receiver in the form of an induction coil 54, and charging circuitry 56 for charging the battery from the coil. In the present embodiment the communications module 16 has a battery alarm function configured to be activated if battery charge declines to a threshold value, as an indication to the user 18 that the module requires charging. The battery alarm function may be configured to emit an audible signal and/or to provide a haptic signal (such as vibration perceptible through the contact of the module with the skin of the user 18) and/or to transmit an alarm signal through the wide area network. In some embodiments each of the auscultation modules 14 may be provided with a respective onboard battery alarm. However, in the present embodiment the auscultation modules 14 are each configured, when their battery charge declines to a threshold value, to send a battery alarm signal to the communications module 16, which is configured in response to provide the required battery alarm.

The use of wireless charging and RF communication avoids a need to provide the communications module 16 with any external port for electrical connection, which could provide a route for ingress of water or other contaminants to the module, and which could be vulnerable to physical damage.

In the present embodiment the communications module 16 is releasably carried upon the wearable sensor carrier 12, in the same general manner described above with reference to the auscultation modules 14.

The communications module 16 may have one or more on-board sensors. In the present embodiment it comprises a motion sensor, and more specifically a BCG (ballistocardiographic) sensor 60. This may comprise a MEMs-type accelerometer. The output of the BCG sensor 60 may be used to cause the wearable auscultation device 10 to automatically activate when donned by the user 18. In the present embodiment, the communications module 16, when initially activated, adopts a standby state in which it accepts a data stream from the BCG but is otherwise largely dormant. If the BCG data stream changes from a resting pattern, the communications module 16 moves to a ready state in which it processes the BCG data stream to establish whether it represents a heartbeat. This processing may comprise application of a Fourier transform to the BCG data stream, to identify frequency components indicative of a heartbeat. If a heartbeat is detected, the communications module 16 enters an active state. If no heartbeat is detected, it returns to the standby state. While in the active state, the communications module 16 continues to process the BCG data stream to determine whether a heartbeat is detected, and is configured to return to the standby state in the event that no heartbeat is detected. The BCG data stream, and/or data such as heart rate derived from it, is logged over time for reporting and analysis.

The communications module 16 and/or the wearable auscultation device 10 as a whole may comprise further sensors. For example, either may comprise one or more sensors responsive to ambient conditions. In this way the effect of ambient conditions on the user's state of health may be determined. Such sensors may include sensors responsive to pollutants in the air. They may comprise on or more sensors responsive to ambient temperature.

In the active state, the communications module 16 polls each of the auscultation modules 14 in turn for audio data, which is logged in the memory 42 and, connectivity permitting, is output through the modem 46 along with the BCG sensor data. A sensor for monitoring of blood oxygen saturation may be included. This may take the form of a pulse oximeter.

The communications module 16 also receives metadata from the auscultation modules 14, which may for example report their level of battery charge, any error codes, and so on. Loss of connectivity between the communications module 16 and any of the auscultation modules 14 may generate an error code and may also cause the communications module 16 to return to standby mode. Metadata may be reported through the modem 46, for remote monitoring and/or analysis.

Functional units of the auscultation module 14 are represented in FIG. 6. The auscultation module 14 comprises an on-board electrical power supply in the form of a rechargeable battery 70. In present embodiments a lithium-ion battery is used. Charging could in principle be made through electrical connections, e.g. comprising a socket for a charging lead, but in the present embodiment wireless inductive charging is used. The auscultation module 14 comprises an induction coil 72 and charging circuitry 74 for charging the battery from the coil. The use of wireless charging and RF communication avoids a need to provide the auscultation module 14 with any external port for electrical connection, which could provide a route for ingress of water or other contaminants to the module, and which could be vulnerable to physical damage.

The auscultation module 14 comprises an on-board processor 76 and associated memory 78, and a Bluetooth® interface 80 for data exchange with the communications module 16. It further comprises an auscultation microphone 82 and an ambient noise microphone 84. The word “microphone” as used in the present description and in the appended claims refers to any sensor device capable of sensing acoustic vibrations and generating a corresponding acoustic data stream. That is, it refers to an acoustic transducer. In the present embodiment the microphones 80, 82 are MEMs-type devices, but subject to packaging constraints the invention could for example be implemented using fibre optic microphones, dynamic microphones, electret microphones, ribbon microphones, laser microphones, condenser microphones, cardioid microphones, crystal microphones, accelerometers of any suitable type, and so on.

The auscultation microphone 82 faces toward the contact face 39, and hence in use toward the skin of the user 18, and is acoustically coupled to the user 18, as will be explained hereinafter. The ambient noise microphone 84 faces away from the user 18 in use, to capture ambient noise.

In certain embodiments of the present invention, the auscultation module 14 comprises an elastomer body 86 which contains and houses the functional components of the module. The elastomer body 86 may, in the finished article, be a unitary body of elastomer, even if it is cast in a number of individual steps. It may closely embrace the module's functional components. The functional components may be embedded in the elastomer body 86.

A process suitable for manufacture of the auscultation module 14 will now be explained. The elastomer body 86 is formed by casting of a setting elastomer material in a mould 88. A single-piece mould could in principle be used, but in the present embodiment the mould comprises a tray portion 90 and a frame portion 92 which are assembled to one another to form the mould cavity 94, as depicted in FIG. 7C. This two-part mould formation is used to facilitate mould release. In the moulding process, the mould 88 is horizontally oriented and liquid settable elastomer is poured to partially fill the mould to a line 95 seen in FIG. 8. A circuit board 96 carrying the functional components of the auscultation module 14 is then placed in the mould cavity, upon the already-poured elastomer, and the mould cavity is then filled to the required level with further liquid settable elastomer. Silicone is a suitable elastomer material and is used in the present embodiment. Other elastomeric materials may however be used. After curing, the process provides an elastomer body 86 which is unitary in nature, as depicted in FIG. 10. The functional components of the auscultation module 14 are wholly contained therein, and are physically protected from shocks or impacts, and from contamination.

The communications module 16 can be manufactured in a broadly similar manner, with its functional components being carried on a circuit board around which a protective body of settable elastomer is moulded.

The microphones 82, 84 are themselves carried upon the circuit board 96 and encased in the elastomer body 86. Sounds waves are conducted to the microphones through the elastomer material of the body 86. To put this another way, the microphone is acoustically coupled to the skin of the user 18 through the elastomer material. In respect of the auscultation microphone 82, this is in fact highly advantageous. The acoustic impedance of the elastomer material may be chosen to closely correspond to that of the adjacent tissue of the user 18. In use, the auscultation module 14 rests with its contact face 39 against the user's skin. At this interface, the sounds to be detected are inevitably partially transmitted and partially reflected. By matching acoustic impedance at the interface, transmission is maximised and signal loss minimised.

In accordance with the present invention, an acoustic element 98 is placed in the path taken by sound waves through the elastomer body 86 to the auscultation microphone 82. It comprises a material having a different acoustic impedance from the elastomer body 86. This may be a non-elastomer. In the present embodiment it is acrylonitrile butadiene styrene (ABS). In the present embodiment the acoustic element 98 has the form depicted in FIGS. 11 and 12, with a hollow body in the form of a frustum 100 of a cone which converges toward the auscultation microphone 82. The hollow body forms an acoustic chamber, which in the present embodiment is elastomer filled. The sensing part of the auscultation microphone is not seen in FIG. 11 but it lies on the axis of, and beneath, the cone frustum 100. A support structure 102 is used during manufacture to raise the cone 100 from the circuit board 96, and to receive the auscultation microphone 82 in the correct position. The acoustic element 98 is embedded in the elastomer body 86 and in intimate contact with it. During manufacture, the cone 100 is filled with elastomer material which is then allowed to set, and is then placed on the circuit board 96 (FIG. 12) prior to the casting process described above. The elastomer with which the cone 100 is filled may be the same material used in the remainder of the moulding. Alternatively, it may be a different material. In particular, the two different elastomers may have different acoustic properties. It may be for example that both comprise the same settable elastomer material, but one contains an admixture of a density-modifying component such as bubbles or microballoons.

The acoustic element 98 has a geometric shape designed to selectively amplify clinically useful elements of the signal. A particular challenge in processing of the audio signals lies in distinguishing between sounds (from breathing and the circulatory system) that originate in the user's body and form the desired signal, and unwanted sounds resulting from movement of the auscultation modules 14 relative to the skin of the user, which have no information value and must be regarded as noise in the signal. Conventional stethoscopes use an air-chamber to convert the vibrations on the chest wall into a meaningful sound pressure wave. This air-coupler generally takes the form of a cone, with the cone material having a significantly different acoustic impedance to air. This formulation helps to both reduce the signal attenuation when travelling from the chest wall to the sensor and to amplify the acoustic spectrum around the acoustic frequencies of clinical importance. Studies have explored optimising the design of the air-chamber to reduce signal attenuation through the air. Air chambers of less depth are known to cause less signal attenuation, but, equally, the amplitude of the initial signal is proportional to the area of the chest piece at the torso-end of the air chamber. Therefore, a cone optimises the conduction of the signal through the air chamber by maximising the area-to-volume ratio. An air-coupler is a necessary fact of the acoustic stethoscope, as, in order to be detectable by human ears, the pressure wave generated at the surface of the chest must be conveyed directly through the air to the ears. This has had a substantial impact on the design choices of electronic stethoscopes, but, more recently, contact technologies such as piezoelectric transducers and accelerometers have been used to capture the signal at the chest wall. Considerations around removing the poor conduction of the signal through an air medium have become more important. On the other hand, these technologies are very vulnerable to friction noise, as, invariably, such contact technologies are unable to isolate the chest wall vibrations from sounds created by movements of the sensor across the chest wall. One solution is to fix the sensor in place on the user's skin, but this is not acceptable for long term monitoring, where users must be able to remove and apply sensors themselves and do this comfortably. Therefore, particularly when considering long monitoring windows through which a user might be walking around or conducting normal day-to-day activities, friction noise becomes a key problem.

One of the advantages of an air coupler is reducing this friction noise by scattering sounds which are not generated perpendicularly to the air chamber. A coupler mechanism having this effect would therefore be useful in the application of long-term respiratory monitoring. However, air couplers of the type found in conventional stethoscopes are too bulky for this application due to the size of the chamber necessitated by the signal attenuation caused by the signal conduction through this medium. On the other hand, other materials can be used as the medium through which the signal travels to the sensor. In embodiments of the invention, an acoustic chamber is created through a geometric shape (for example, a cone or cylinder) whose volume is of a different acoustic impedance to its surface, through which the signal is conducted without significant attenuation. By conducting the signal through this “impedance-coupler” rather than directly through the medium, the benefits of acoustic-coupling can be realised without the drawbacks of air-coupling.

In the illustrated embodiment, the acoustic element 98 is found in practice to selectively amplify noises emanating from the chest wall relative to friction noise, improving signal quality.

Another factor contributing to noise in the audio signal is ambient noise in the user's environment. The ambient noise microphones 84 are used to cancel ambient noise. This may be done on the assumption that the ambient noise microphone 84 receives only ambient noise, while the auscultation microphone receives both ambient noise and the audio signal from the user's body, so that subtracting one from the other leaves (to an approximation) only the desired audio signal. This subtraction of one signal from the other could be made by each individual auscultation module 14, but in the present embodiment this aspect of the signal processing is carried out by the communications module 16, which receives ambient noise data streams from each of the auscultation modules 14, and deducts an average ambient noise signal obtained therefrom, from the individual audio streams from the auscultation modules 14. Ambient noise cancellation is important not only for the sake of signal quality, but also in the interests of privacy: since the wearable auscultation device 10 will be used to record sounds over a protracted period, often at the home of the user, it is desirable that no conversation or other potentially private audible events should be recorded and transmitted.

The sensor units 14 and the communications module 16 require periodic battery charging. This may be achieved by removing the modules form the wearable sensor support 12 and placing each in the vicinity of a suitable wireless charging device. Alternatively, the user may be provided with a charging device comprising a hanger, support or mannequin which is for supporting the wearable sensor support 12 and which is provided with a number of wireless charging devices at appropriate locations, so that placing the wearable auscultation device on the charging device aligns the modules 14, 16 with the charging devices, allowing each to be inductively charged.

The audio data streams from the auscultation modules 14, along with data streams from any other sensors of the wearable auscultation device 10, may be exploited in a variety of different ways.

The audio data may be used by a clinician or other individual through an application running on a suitable computing device 110, which may be a mobile phone (cell phone)—see FIG. 5. The clinician's computing device may for example receive the audio data streams (and/or metadata derived from them) from the server 44, via the internet 112 following suitable authentication processes intended to ensure that the data is not inappropriately distributed. This may be done in real time, e.g. in the context of a real time audio or video consultation with the patient 18, and/or the clinician may review recorded data. The multiple auscultation modules 14 allow the clinician to listen to all of the sites (s)he might access with a stethoscope, but since the wearable auscultation device 10 can be worn for protracted periods, the clinician is able e.g. to monitor variations taking place in the user's normal day, or to search in the data for exceptional events that might be indicative of intermittently occurring clinical conditions.

The audio data may be subject to digital processing for a range of diagnostic or health assessment and monitoring purposes. Computer-based techniques may be used to obtain diagnostic data and other data from the data streams provided by the wearable auscultation device. For example, trained machine-learning systems may be used to identify adventitious sounds potentially indicative of respiratory or other disorders. Real time monitoring may be carried out to provide alerts in the event that the user has an episode which requires treatment.

FIGS. 13 to 15 depict features of a further auscultation module 14a according to an embodiment of the invention. The auscultation module 14a can be used in a version of the auscultation device 10 which is in other respects similar to the above-described embodiments. In this embodiment the auscultation microphone receives the acoustic signal via a body 120 of gas (FIG. 5). This is desirable in relation to certain types of transducer (microphone), designed to receive audio signals through the air. The gas is air in the present embodiment although other gases could be used, especially to provide the body 120 of gas with a desired acoustic impedance. The body 120 of gas is disposed in a cavity 122 within elastomer body 86a of the auscultation module 14a, and communicates with auscultation microphone 82a, which is omitted from FIG. 15 but seen in FIG. 16. The auscultation microphone 82a is seen in FIG. 16 to sit atop circuit board 96a of the auscultation module 14a. But alternatively it may be mounted on the underside of the circuit board 96a, communicating with the cavity 122 via a through-going opening in the circuit board 96a. This through-going opening (not shown) may be of small lateral dimension, of the order of 0.5 mm, commensurate with the size of the active part of the microphone 82a, although this size may differ in other embodiments.

The further auscultation module 14a comprises an acoustic element 98a disposed in the acoustic pathway through the elastomer body 86a to the auscultation microphone 82a. As in the first embodiment, the acoustic element 98a is embedded in the elastomer body 86a and is in intimate contact with it. It has a form which converges along the acoustic pathway in the direction toward the microphone 82a. It defines an internal space 124 through which sound is conducted to reach the auscultation microphone 82a. The internal space 124 converges along the acoustic pathway in the direction toward the microphone 82a. The cross-section of the internal space 124 progressively reduces along the acoustic pathway in the direction toward the microphone 82a. The convergent form of the acoustic element 98a serves to focus and concentrate sound energy upon the auscultation microphone 82a. In the present embodiment the acoustic element 98a comprises a frustum 100a of a cone having an axial through-going opening 126 in the acoustic pathway to the auscultation microphone 82a. The acoustic element comprises a material having a different acoustic impedance from the elastomer body 86a. The material may be a non-elastomer and may be ABS.

To form the cavity 122 in the elastomer body 86a, the auscultation module 14a is made using a cavity member 128. In the present embodiment the cavity member 128 comprises the same elastomer material as the elastomer body 86a, so that when the elastomer body 86a has been poured and set, in essentially the same manner already described, the cavity member 86a forms part of the elastomer body 86a, without any pronounced change of acoustic impedance at its boundary 130 (FIG. 15). Note that while in FIG. 15 the cavity member 128 is hatched differently from the remainder of the elastomer body 86a, these two regions may be fused together and essentially continuous in terms of material properties. The cavity member 128 comprises in the present embodiment a lower pedestal portion which locates in a complementary opening in a baseboard 132, and an upstanding nipple 134 which is received in and located by the opening 126, to locate the elastomer body 86a— and hence the cavity 122—with respect to the acoustic element 98a.

The cavity member 128 seats through its underside upon the circuit board 96a, forming a seal against egress of air from the cavity 122. Threaded fasteners 136 are passed through aligned openings in tabs 138, 140 of the acoustic element 98a and of the baseboard 132, and through openings 142 in the circuit board 96a, to mount these parts to the board. Tightening these fasteners during assembly compresses the cavity member 128 and so reduces the volume of the cavity 122, so that gas pressure in the cavity 122 is higher than atmospheric pressure. This raises the acoustic impedance of the gas in the cavity 122, which is desirable in terms of transmission of the acoustic signal. The path length through the cavity 122 is short, minimising transmission losses in this region.

Test have demonstrated that the presence of the convergent acoustic element 98a improves detection of the desired signal—sounds emitted from the body—whilst also reducing unwanted noise due to friction/shear at the interface between the auscultation module 14a and the body.

1. A wearable auscultation device comprising a wearable sensor support configured to be worn on a user's torso and to carry a plurality of auscultation modules, positioning the plurality of auscultation modules in use at respective auscultation sites on the torso and retaining the plurality of auscultation modules in contact with the user's skin, wherein at least one of the auscultation modules comprises:
a body of elastomer material providing a contact face for contact with the user's skin;
an auscultation microphone directed toward the contact face to sense sound waves conducted through the elastomer material, and
an acoustic element having an acoustic impedance different from the acoustic impedance of the elastomer material, the acoustic element being embedded in the body of elastomer material between the contact face and the auscultation microphone and defining an acoustic chamber which converges in the direction toward the auscultation microphone.

2. The wearable auscultation device as claimed in claim 1, wherein the acoustic chamber is elastomer filled.

3. The wearable auscultation device as claimed in claim 1, wherein the acoustic element includes a hollow conical form which converges in the direction toward the auscultation microphone.

4. The wearable auscultation device as claimed in claim 1, wherein said auscultation module further includes the following functional units:
a processor;
a memory;
a data interface for outputting an audio data stream; and
an electrical battery.

5. The wearable auscultation device as claimed in claim 4, wherein the functional units of the auscultation module are contained and embedded in the body of elastomer material.

6. The wearable auscultation device as claimed in claim 4, wherein the data interface is an unwired data interface which transmits through the elastomer material.

7. (canceled)

8. (canceled)

9. (canceled)

10. (canceled)

11. The wearable auscultation device as claimed in claim 1, wherein said auscultation module further includes an ambient noise microphone directed away from the contact surface.

12. The wearable auscultation device as claimed in claim 11, the wearable auscultation device is configured to cancel ambient noise from an audio data stream output from the auscultation microphone.

13. The wearable auscultation device as claimed in claim 1, wherein the auscultation modules are removably attachable to the wearable sensor support.

14. The wearable auscultation device as claimed in claim 14, wherein the wearable sensor support includes a plurality of auscultation module couplings each configured to receive and mount one respective auscultation module.

15. The wearable auscultation device as claimed in claim 14, wherein each of the auscultation module couplings includes an aperture to receive the auscultation module and to engage its periphery.

16. The wearable auscultation device as claimed in claim 1, further comprising a sensor for detecting a heartbeat.

17. The wearable auscultation device as claimed in claim 17, wherein the wearable auscultation device is configured to move from a dormant state to an active state upon detection of a heartbeat.

18. The wearable auscultation device as claimed in claim 1, wherein the auscultation microphone communicates with a gas-filled chamber formed in the body of elastomer material.

19. The wearable auscultation device as claimed in claim 18, wherein the acoustic element includes a through-going opening arranged such that sound waves are able to pass through the through-going opening to reach the auscultation microphone.

20. (canceled)

21. An auscultation module comprising a body of elastomer material providing a contact surface for placement against skin of a user and containing:
an auscultation microphone directed toward the contact surface, so that in use the elastomer material conducts sound emitted by the user to the auscultation microphone; and
an acoustic element having an acoustic impedance different from the acoustic impedance of the elastomer material, the acoustic element being embedded in the body of elastomer material between the contact face and the auscultation microphone and defining an acoustic chamber which converges in the direction toward the auscultation microphone.

22. The auscultation module as claimed in claim 21, wherein the acoustic element includes a hollow conical form which converges in the direction toward the auscultation microphone.

23. A method of manufacture of an auscultation module, the method comprising:
providing a mould;
arranging in the mould functional units of the auscultation module including an auscultation microphone;
inserting into the mould a liquid-form settable elastomer; and
setting of the elastomer, so that the functional units of the auscultation module are contained and embedded in a resulting elastomer body.

24. The method as claimed in claim 23, further comprising:
providing some or all of the functional units on a circuit board;
partially filling the mould with the liquid-form settable elastomer;
placing the circuit board upon the poured elastomer; and
inserting further liquid-form settable elastomer over the circuit board.

25. The method as claimed in claim 23, further comprising placing an acoustic element between the auscultation microphone and a surface of the mould, so that the acoustic element is embedded in the elastomer body.
